(19) United States
(12) Patent Application Publication
(10) Pub. No.: US 2025/0375375 A1
(43) Pub. Date: Dec. 11, 2025

(54) ANTIMICROBIAL GELS

(71) Applicant: ROYAL MELBOURNE INSTITUTE OF TECHNOLOGY, Melbourne, Victoria (AU)

(72) Inventors: Saffron BRYANT, Melbourne, Victoria (AU); Aaron ELBOURNE, Melbourne, Victoria (AU); Sumeet WALIA, Melbourne, Victoria (AU); Miyah AWAD, Melbourne, Victoria (AU); Gary BRYANT, Melbourne, Victoria (AU); Tamar GREAVES, Melbourne, Victoria (AU)

(21) Appl. No.: 18/876,675

(22) PCT Filed: Jun. 23, 2023
(86) PCT No.: PCT/AU2023/050578

§ 371 Date: Dec. 19, 2024

(30) Foreign Application Priority Data
Jun. 24, 2022 (AU) 2022901764

(51) Int. Cl. A61K 9/06; A61K 9/70; A61K 33/24; A61K 33/242; A61K 33/243; A61K 33/30; A61K 33/34; A61K 33/38; A61K 47/10; A61K 47/18

(52) U.S. Cl. CPC A61K 9/06; A61K 9/7007; A61K 33/24; A61K 33/242; A61K 33/243; A61K 33/30; A61K 33/34; A61K 33/38; A61K 47/10; A61K 47/18; A61K 47/186

(57) ABSTRACT

The present disclosure relates generally to antimicrobial gels. In particular, the present disclosure relates to an antimicrobial gel comprising a eutectic solvent and a gelling agent which can be used as a drug delivery vehicle for antimicrobial agents, including for the topical delivery of antimicrobial agents to the skin of a subject. The present disclosure also relates to processes for preparing the antimicrobial gel, and to methods, uses and compositions comprising the antimicrobial gel.

### FIELD

The present disclosure relates generally to antimicrobial gels. In particular, the present disclosure relates to an antimicrobial gel comprising a eutectic solvent and a gelling agent which can be used as a drug delivery vehicle for antimicrobial agents, including for the topical delivery of antimicrobial agents to the skin of a subject. The present disclosure also relates to processes for preparing the antimicrobial gel, and to methods, uses and compositions comprising the antimicrobial gel.

### BACKGROUND

Microorganisms present significant challenges to wound healing and increase morbidity and mortality. Treatment of wounds, such as burns, cuts, and puncture wounds, can be difficult in part due to possible infections from microorganisms, and the rise in incidence of superinfections and multiple drug resistant microorganisms. Wound dressings have been used to promote healing, where recent studies have shown that a moist environment helps to promote healing and subsequent patient recovery.

Hydrogel wound dressings have been extensively studied to date for use as wound dressings. Hydrogels are cross-linked hydrophilic polymers that absorb and retain significant amounts of water, which can keep the underlying environment moist when applied to the skin. By absorbing and retaining water, hydrogels also permit the transdermal delivery of therapeutic compounds through the skin. However, owing to their high water content, hydrogels can evaporate and dry out quickly which necessitates regular removal and reapplication when used as wound dressings which can cause more pain/injury, for example, to burns victims. Furthermore, hydrogels are often limited to water-soluble drugs thus significantly limiting the microorganisms they can target. Accordingly, there is a need for alternative or improved wound dressings that can address one or more of the above problems and/or provide the public with a useful alternative.

It will be understood that any prior art publications referred to herein do not constitute an admission that any of these documents form part of the common general knowledge in the art, in Australia or in any other country.

### SUMMARY

The present inventors have undertaken research and development into gels, particularly eutectogels. The gel comprises a eutectic solvent and gelling agent, together with a therapeutic agent. The gel can comprise one or more antimicrobial agents. According to some embodiments or examples described herein, the antimicrobial gel can be used to topically deliver antimicrobial agents to the skin. The present inventors have surprisingly found that antimicrobial gels formed using eutectic solvents do not dry out over a prolonged period of time. When used as wound dressings, this minimizes the need for ongoing reapplication as the antimicrobial gels retain the eutectic solvent and remain “moist” (i.e. do not dry out) throughout the entire wound treatment period. This leads to improving overall care for patients, whilst allowing for the delivery of a wide range of antimicrobial agents.

In one aspect, there is provided a gel, comprising: a) a eutectic solvent comprising a mixture of at least one hydrogen bond acceptor and at least one hydrogen bond donor; b) a gelling agent; and c) a therapeutic agent.

In another aspect, there is provided an antimicrobial gel, comprising: a) a eutectic solvent comprising a mixture of at least one hydrogen bond acceptor and at least one hydrogen bond donor; b) a gelling agent; and c) an antimicrobial agent.

In another aspect, there is provided an antimicrobial gel, comprising: a) a deep eutectic solvent (DES) comprising a mixture of at least one hydrogen bond acceptor and at least one hydrogen bond donor, wherein the molar ratio of the hydrogen bond acceptor to the hydrogen bond donor in the eutectic solvent is at or near the eutectic point of the solvent; b) a gelling agent; and c) an antimicrobial agent.

In another aspect, there is provided an antimicrobial gel, comprising: a) a eutectic solvent comprising a mixture of at least one hydrogen bond acceptor and at least one hydrogen bond donor; b) a gelling agent; and c) an antimicrobial agent, wherein the concentration of eutectic solvent (% w/w) is between about 70 to about 99.9 based on the total weight of the antimicrobial gel.

In another aspect, there is provided an antimicrobial gel, comprising: a) a eutectic solvent comprising a mixture of at least one hydrogen bond acceptor and at least one hydrogen bond donor; b) a gelling agent; and c) an antimicrobial agent; wherein the antimicrobial gel has a water content (% w/w) of less than about 5 based on the total weight of the antimicrobial gel.

In another aspect, there is provided an antimicrobial gel, comprising: a) a eutectic solvent comprising a mixture of at least one hydrogen bond acceptor and at least one hydrogen bond donor; b) a gelling agent; and c) an antimicrobial agent; wherein the antimicrobial gel is substantially free of water and/or an additional organic solvent.

In another aspect, there is provided a process for preparing an antimicrobial gel as described above, comprising mixing a gelling agent and a eutectic solvent under conditions effective to form a gel, and wherein the process comprises mixing an antimicrobial agent with the eutectic solvent and gelling agent or contacting the gel with the antimicrobial agent under conditions effective to incorporate the antimicrobial agent into the gel.

In another aspect, there is provided a topical dressing comprising an antimicrobial gel as described above.

In another aspect, there is provided a method of treating and/or preventing a skin condition in a subject in need thereof, the method comprising applying a therapeutically effective amount of an antimicrobial gel as described above or a topical dressing as described above to the skin of the subject.

In another aspect, there is provided use of an antimicrobial gel as described above or a topical dressing as described above for use in treating and/or preventing a skin condition.

In another aspect, there is provided use of an antimicrobial gel as described above or a topical dressing as described above for the manufacture of a medicament for treating and/or preventing a skin condition in a subject.

These and other aspects and embodiments relating to the present disclosure are further described herein. It will be appreciated that any one or more of the examples, aspects or embodiments described herein is taken to apply mutatis mutandis to each and every other example, aspect or embodiment unless specifically stated otherwise. The present disclosure is not to be limited in scope by the specific examples described herein, which are intended for the purpose of exemplification only.

### BRIEF DESCRIPTION OF THE DRAWINGS

Preferred embodiments of the present disclosure are further described and illustrated as follows, by way of example only, with reference to the accompanying drawings in which:

FIG. 1: Shows examples of the chemical structures of the deep eutectic solvent components that can be used to prepare the antimicrobial gels.

FIG. 2: Shows the average annular radius of the zone of inhibitions of P. aeruginosa, MRSA, and C. albicans exposed to deep eutectic gels containing Ag NP with particle size of 10 nm at concentrations between 0 mg/g and 0.05 mg/g. Error bars are based on the standard deviation of three replicates.

FIG. 3: Shows the average annular radius of the zone of inhibitions of P. aeruginosa exposed to deep eutectic gels containing Ag NP of particle sizes 10 nm, 20 nm, and 100 nm, at a concentration of 0.05 mg/g. Error bars are based on the standard deviation of three replicates.

FIG. 4: Shows the average number of CFU/mL of (a) P. aeruginosa, (b) MRSA, and (c) C. albicans, and average reduction (%) in CFU/mL of (d) P. aeruginosa, (e) MRSA, and (f) C. albicans, after 3, 6, 24, and 48 hours of exposure to eutectogels containing NP with a particle size of 10nm at concentrations ranging from 0.0 mg/g to 0.05 mg/g. For (a), (b), and (c), error bars are based on the standard deviation of three replicates, where some are smaller than the symbols. For (d), (e), and (f), colour codes are as follow: light grey—1 log reduction (90%), dark grey—2 log reduction (99%), and—3 log reduction (99.9%); dark grey and bold letters—3 log reduction (99.99%). Values given to two decimal places, with the exception of values above 99.99%.

FIG. 5: Shows the average reduction (%) of CFU/mL of (a) P. aeruginosa, (b) MRSA, and (c) C. albicans after 3, 6, 24, and 48 hours of exposure to the deep eutectic gels containing Ag NP at concentrations ranging from 0.0 mg/g to 0.05 mg/g. Reduction in CFU/mL is normalised to the control. Error bars are based on the standard deviation of three replicates.

FIG. 6: Shows the average reduction (%) of CFU/mL of P. aeruginosa, MRSA, and C. albicans after (a) 24 hours and (b) 48 hours of exposure to the deep eutectic gels containing Ag NP at concentrations ranging from 0.0 mg/g to 0.05 mg/g. Reduction in CFU/mL is normalised to the control. Error bars are based on the standard deviation of three replicates.

FIG. 7: Shows the zone of inhibition tests for eutectogels loaded with black phosphorous nanoparticles against MRSA and P. aeruginosa. The graph on the right hand side shows the zones of inhibition against both organisms, using ‘treatment A’ i.e. well technique.

FIG. 8: Shows an overview of the A) colony counting and B) zone of inhibition (ZOI) protocol used. It further shows C) reduction in cell count for the bare and antimicrobial loaded gels as a log10 reduction of control after 24 hour exposure; and D) the ZOI diameters for the eutectogels. The mean ±SD values are shown, and * indicates significant difference compared to the bare gel (P<0.05) using ANOVA one-way analysis followed by Tukey's multiple comparison test. *P<0.05, **P<0.01, ***P<0.001 and ****P<0.0001.

FIG. 9: Representative phase diagram for choline chloride: urea.

### DETAILED DESCRIPTION

The present disclosure describes the following various non-limiting embodiments, which relate to investigations undertaken to identify new and improved gels, particularly eutectogels, which can be used to topically deliver therapeutic agents, such as antimicrobial agents, to the skin for treating various skin conditions. According to some embodiments or examples, owing to their ease of synthesis, reduced water content and/or properties, the antimicrobial gels described herein are capable of being manufactured at large scale and can be easily handled/stored and/or applied to the skin. Other applications and advantages associated with the antimicrobial gels, topical dressings, compositions, kits, processes, methods, and uses are also described herein.

### Terms

In the following description, reference is made to the accompanying drawings which form a part hereof, and which is shown, by way of illustration, several embodiments. It is understood that other embodiments may be utilized and structural changes may be made without departing from the scope of the present disclosure.

With regards to the definitions provided herein, unless stated otherwise, or implicit from context, the defined terms and phrases include the provided meanings. Unless explicitly stated otherwise, or apparent from context, the terms and phrases below do not exclude the meaning that the term or phrase has acquired by a person skilled in the relevant art. The definitions are provided to aid in describing particular embodiments, and are not intended to limit the claimed invention, because the scope of the invention is limited only by the claims. Furthermore, unless otherwise required by context, singular terms shall include pluralities and plural terms shall include the singular.

All publications discussed and/or referenced herein are incorporated herein in their entirety.

Any discussion of documents, acts, materials, devices, articles or the like which has been included in the present specification is solely for the purpose of providing a context for the present disclosure. It is not to be taken as an admission that any or all of these matters form part of the prior art base or were common general knowledge in the field relevant to the present disclosure as it existed before the priority date of each claim of this application.

Throughout this disclosure, unless specifically stated otherwise or the context requires otherwise, reference to a single step, composition of matter, group of steps or group of compositions of matter shall be taken to encompass one and a plurality (i.e., one or more) of those steps, compositions of matter, groups of steps or groups of compositions of matter. Thus, as used herein, the singular forms “a”, “an” and “the” include plural aspects unless the context clearly dictates otherwise. For example, reference to “a” includes a single as well as two or more; reference to “an” includes a single as well as two or more; reference to “the” includes a single as well as two or more and so forth.

Those skilled in the art will appreciate that the disclosure herein is susceptible to variations and modifications other than those specifically described. It is to be understood that the disclosure includes all such variations and modifications. The disclosure also includes all of the examples, steps, features, processes, antimicrobial gels, topical dressings, compositions, kits, processes, methods, and usesreferred to or indicated in this specification, individually or collectively, and any and all combinations or any two or more of said steps or features.

The term “and/or”, e.g., “X and/or Y” shall be understood to mean either “X and Y” or “X or Y” and shall be taken to provide explicit support for both meanings, or for either meaning.

As used herein, the term “about”, unless stated to the contrary, typically refers to a range of up to +/−10% of the designated value, and includes smaller ranges therein, for example +/−5%, or +/−1% of the designated value.

It is to be appreciated that certain features that are, for clarity, described herein in the context of separate embodiments, may also be provided in combination in a single embodiment. Conversely, various features that are, for brevity, described in the context of a single embodiment, may also be provided separately or in any sub-combination.

Throughout the present specification, various aspects and components of the invention can be presented in a range format. The range format is included for convenience and should not be interpreted as an inflexible limitation on the scope of the invention. Accordingly, the description of a range should be considered to have specifically disclosed all the possible sub-ranges as well as individual numerical values within that range, unless specifically indicated. For example, description of a range such as from 1 to 5 should be considered to have specifically disclosed sub-ranges such as from 1 to 3, from 1 to 4, from 1 to 5, from 2 to 4, from 2 to 5, from 3 to 5 etc., as well as individual and partial numbers within the recited range, for example, 1, 2, 3, 4, 4.5, 4.75, and 5, unless where integers are required or implicit from context. This applies regardless of the breadth of the disclosed range. Where specific values are required, these will be indicated in the specification.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

The reference to “substantially free” generally refers to the absence of that compound or component in a composition, such as in the antimicrobial gel or eutectic solvent for example, other than any trace amounts or impurities that may be present, for example in an amount (% w/w) based on the total weight of the antimicrobial gel or eutectic solvent of less than about 1, 0.1, 0.01, 0.001, or 0.0001. An example of a possible impurity is water.

The reference to “% w/w” refers to the proportion of a particular component within a composition, as measured by weight. “% w/w” may also be referred to as “wt %” or “weight %”. The reference to “mg/g” refers to the mass of a particular component within a composition, based on the total mass of the composition.

### Antimicrobial Gels

According to some examples or embodiments described herein, the present inventors have developed an antimicrobial gel. The antimicrobial gel comprises a eutectic solvent which is gelled using a gelling agent. Owing to the presence of the eutectic solvent, the antimicrobial gel may also be called a “eutectogel” or “eutectic gel”, with the terms being used interchangeably.

The present disclosure provides an antimicrobial gel comprising: a) a eutectic solvent comprising a mixture of at least one hydrogen bond acceptor and at least one hydrogen bond donor; b) a gelling agent; and c) an antimicrobial agent.

In one embodiment, the antimicrobial gel comprises or consists of: a) a eutectic solvent comprising or consisting of a mixture of at least one hydrogen bond acceptor and at least hydrogen bond donor; b) a gelling agent; c) an antimicrobial agent; and optionally water and/or one or more additives.

In a related embodiment, the antimicrobial gel comprises or consists of: a) a eutectic solvent comprising or consisting of a mixture of at least one hydrogen bond acceptor and at least one hydrogen bond donor, in an amount of between about 80% w/w to about 99.9% w/w; b) a gelling agent in an amount of between about 0.1% w/w to about 5% w/w; and c) an antimicrobial agent in an amount of between about 0.0001% w/w to about 1% w/w, based on the total weight of the antimicrobial gel; and optionally water and/or one or more additives.

In one embodiment, the antimicrobial gel consists essentially of a) a eutectic mixture of at least one hydrogen bond acceptor and at least one hydrogen bond donor; b) a gelling agent; and c) an antimicrobial agent.

In a related embodiment, the antimicrobial gel comprises a gel which is obtainable by mixing a eutectic solvent comprising a mixture of at least one hydrogen bond acceptor and at least one hydrogen bond donor with a gelling agent, wherein the gel comprises an antimicrobial agent.

The antimicrobial agent, eutectic solvent, gelling agent, water content and one or more additives are described herein.

### Antimicrobial Agent

The antimicrobial gel comprises an antimicrobial agent. As used herein, the term “antimicrobial agent” refers to a natural or synthetic substance that kills or inhibits the growth of microorganisms such as bacteria, fungi, viruses and parasites. For example, antibiotics are used against bacteria, antifungals are used against fungi, antivirals are used against viruses and antiparasitics are used against parasites. Metallic nanoparticles, such as silver nanoparticles, are also known to have antimicrobial properties. In particular, silver nanoparticles possess a broad spectrum of antibacterial, antifungal, antiviral and antiparasitic properties.

The antimicrobial gel may be used to administer a wide range of antimicrobial agents, including, for example, topically administering antimicrobial agents to the skin. The antimicrobial agent is provided in the gel in an amount effective to exert an antimicrobial effect, for example that can kill or inhibit the growth of microorganisms, including one or more microorganisms that may cause infections in wounds such as burns or cuts on the skin of a subject. Examples of microorganisms that may be treated by the antimicrobial agent include one or more of Pseudomonas aeruginosa (gram-negative bacteria), Staphylococcus auereus (gram-positive bacteria) and Candida albicans (fungi). It will be appreciated that the microorganism that can be effectively treated by the antimicrobial within the gel can vary, and the antimicrobial gel can be tailored to target specific microorganisms depending on the desired topical use. For example, the antimicrobial agent may be specific for one or more microorganisms that are prevalent in a wound, such as a burn or cut. In one embodiment, the antimicrobial agent is a broad-spectrum antimicrobial agent, such as silver nanoparticles.

In one embodiment, the concentration of antimicrobial agent (mg/g) is between about 0.001 to about 100 based on the total weight of the antimicrobial gel (that is, between about 0.0001% w/w to about 10% w/w). In one embodiment, the concentration of antimicrobial agent (mg/g) is at least about 0.001, 0.005, 0.01, 0.02, 0.05, 0.1, 0.5, 1, 5, 10, 50 or 100 based on the total weight of the antimicrobial gel. In one embodiment, the concentration of antimicrobial agent (mg/g) is less than about 100, 50, 10, 5, 1, 0.5, 0.1, 0.05, 0.02, 0.01, 0.005, or 0.001 based on the total weight of the antimicrobial gel. The concentration of the antimicrobial agent may be in a range provided by any two of these upper and/or lower values, for example the concentration of antimicrobial agent (mg/g) may be between about 0.001 to about 10, between about 0.001 to about 5, or between about 0.001 to about 1, based on the total weight of the antimicrobial gel.

It will be appreciated that the mg/g concentration values can also be expressed in terms of % w/w based on the total weight of the antimicrobial gel. In one embodiment, the concentration of antimicrobial agent (% w/w) is between about 0.0001 to about 10 based on the total weight of the antimicrobial gel. In one embodiment, the concentration of antimicrobial agent (% w/w) is at least about 0.0001, 0.0005, 0.001, 0.002, 0.005, 0.01, 0.05, 0.1, 0.5, 1, 5 or 10 based on the total weight of the antimicrobial gel. In one embodiment, the concentration of antimicrobial agent (% w/w) is less than about 10, 5, 1, 0.5, 0.1, 0.05, 0.01, 0.005, 0.002, 0.001, 0.0005, or 0.0001 based on the total weight of the antimicrobial gel. The concentration of antimicrobial agent may be in a range provided by any two of these upper and/or lower values, for example the concentration of antimicrobial agent (% w/w) may be between about 0.0001 to about 1, between about 0.0001 to about 0.5, or between about 0.0001 to about 0.1, based on the total weight of the antimicrobial gel.

In one embodiment, the antimicrobial agent is a topical antimicrobial agent. As used herein, the term “topical” antimicrobial agent refers to natural or synthetic substance that, directly applied to the skin, kills or inhibits the growth of microorganisms such as bacteria, fungi and viruses. Such topical antimicrobial agents need not penetrate the skin and get into the blood stream but rather exert their effect topically on the skin, such as within an open wound, for example, a burn or cut. Accordingly, it will be appreciated that where the antimicrobial gel comprises a topical antimicrobial agent, the gel is not acting as a “drug delivery vehicle” as understood by the person skilled in the art for the transdermal drug delivery of an active pharmaceutical agent across the skin for systemic distribution. This is particularly true for metallic nanoparticles, such as silver nanoparticles, which are not a “drug” but rather antimicrobials.

The antimicrobial agent may be any natural or synthetic substance that can kill or inhibit the growth of microorganisms, including one or more microorganisms that may cause infections (e.g. bacterial, fungal, viral and/or parasitic infections) in wounds such as burns or cuts on the skin of a subject. In one embodiment, the antimicrobial agent is selected from the group consisting of antimicrobial nanoparticles, an antibiotic, an antifungal, an antibacterial, an antiseptic, an antiparasitic or an antiviral, or a combination thereof.

In a preferred embodiment, the antimicrobial agent is a population of antimicrobial nanoparticles. Antimicrobial nanoparticles are increasingly being used to target and treat microorganisms as an alternative to antibiotics, antifungals, antiparasitics and/or antivirals and the like. In one embodiment, the antimicrobial nanoparticles are non-metallic nanoparticles or metallic nanoparticles.

In one embodiment, the antimicrobial nanoparticles are non-metallic nanoparticles. In another embodiment, the non-metallic nanoparticles are black phosphorous nanoparticles.

In one embodiment, the antimicrobial nanoparticles are metallic nanoparticles. In another embodiment, the metallic nanoparticles are selected from the group consisting of silver, gold, nickel, platinum, palladium, cadmium, zinc, and copper nanoparticles, and combinations thereof. In one embodiment, the metallic nanoparticles are silver or gold nanoparticles, or a combination thereof. In one preferred embodiment, the metallic nanoparticles are silver nanoparticles. According to some embodiments or examples described herein, despite the silver nanoparticles being typically water soluble (i.e. provided in an aqueous solution/suspension), the present inventors were able to incorporate them into the substantially non-aqueous eutectic gel environment without causing significant breakdown and/or adverse chemical reactions within the gel.

As used herein, the term “nanoparticle” typically refers to a particle of matter, such as silver nanoparticles, that has a size to 1000 nanometers (nm) or less. The particle size is taken to be the longest cross-sectional diameter across a nanoparticle. For non-spherical nanoparticles, the particle size is taken to be the distance corresponding to the longest cross-section dimension across the particle. The nanoparticle may take the form of flakes, fibres, agglomerates, granules, powders, spheres, pulverized materials or the like, as well as combinations thereof. The nanoparticles may have any desired shape including, but not limited to, cubic, rod like, polyhedral, spherical or semi-spherical, rounded or semi-rounded, angular, irregular, and so forth. The nanoparticle morphology can be determined by any suitable means such as optical microscopy.

In one embodiment, the antimicrobial nanoparticles have a mean average particle size (in nm) of between about 1 to about 1000. In one embodiment, the antimicrobial nanoparticles have a mean average particle size (in nm) of at least about 1, 2, 5, 10, 15, 20, 25, 30, 40, 50, 100, 200, 500 or 1000. In one embodiment, the antimicrobial nanoparticles have a mean average particle size (in nm) of less than about 1000, 500, 200, 100, 50, 40, 30, 25, 20, 15, 10, 5, 2 or 1. The mean average particle size may be in a range provided by any two of these upper and/or lower values, for example the antimicrobial nanoparticles have a mean average particle size (in nm) of between about 1 to about 500, between about 5 to about 200, or between about 10 to about 100.

In a related embodiment, the metallic nanoparticles have a mean average particle size (in nm) of between about 1 to about 1000. In one embodiment, the metallic nanoparticles have a mean average particle size (in nm) of at least about 1, 2, 5, 10, 15, 20, 25, 30, 40, 50, 100, 200, 500 or 1000. In one embodiment, the metallic nanoparticles have a mean average particle size (in nm) of less than about 1000, 500, 200, 100, 50, 40, 30, 25, 20, 15, 10, 5, 2 or 1. The mean average particle size may be in a range provided by any two of these upper and/or lower values, for example the metallic nanoparticles have a mean average particle size (in nm) of between about 1 to about 500, between about 5 to about 200, or between about 10 to about 100.

The antimicrobial agent may be interspersed throughout the antimicrobial gel, including as a dissolved or suspended species. In one embodiment, the antimicrobial gel comprises metallic nanoparticles interspersed on or within the gel.

### Eutectic Solvent

The antimicrobial gel comprises a eutectic solvent. The eutectic solvent is mixed with a suitable amount of gelling agent to form the gel which then acts as a repository for the antimicrobial agent. The eutectic solvent comprises at least a hydrogen bond acceptor and a hydrogen bond donor, which together form a eutectic mixture which inhibits the crystallisation process of one another thereby resulting in a system having a lower melting point (i.e. lower freezing point) than either of the components individually. The term “eutectic solvent” as used herein does not refer exclusively to the solvent mixture having the deepest depression in melting point (i.e. a mixture at or near the eutectic point of the system) such as that seen for deep eutectic solvents (DES), but includes it inter alia. Owing to their relative non-aqueous properties, eutectic solvents are not commonly used for medical purposes. The eutectic solvent may comprise any suitable mixture of hydrogen bond acceptors and hydrogen bond donors effective to form a eutectic mixture. While the eutectic solvent may comprise more than one hydrogen bond acceptor and/or more than one hydrogen bond donor, typically the eutectic solvent comprises a mixture of single hydrogen bond acceptor/donor pair (e.g. a mixture selected from choline chloride and glycerol, choline chloride and urea, or betaine and glycerol etc.). For example, the eutectic solvent may consist of a hydrogen bond acceptor and a hydrogen bond donor. It will be appreciated that in some cases the eutectic solvent may comprise one or more additional additives over and above the hydrogen bond donor(s) and acceptor(s).

As understood by the person skilled in the art, a range of hydrogen bond acceptors may be used to form the eutectic solvent. In one embodiment, the hydrogen bond acceptor is selected from the group consisting of a quaternary ammonium compound, a quaternary phosphonium compound, a quaternary sulfonium compound, a metal salt, an alcohol, and an amino acid, or combinations thereof.

In one preferred embodiment, the hydrogen bond acceptor is a quaternary ammonium compound. Any quaternary ammonium compound may be used. In one embodiment, the quaternary ammonium compound is choline chloride or betaine. In one embodiment, the quaternary ammonium compound is choline chloride. In one embodiment, the quaternary ammonium compound is betaine. In other embodiments, the hydrogen bond acceptor is an alcohol, such as methanol. In other embodiments, the hydrogen bond acceptor is an amino acid, such as L- or D-proline. In one embodiment, the hydrogen bond acceptor is selected from the group consisting of choline chloride, betaine, methanol and proline. It will be appreciated that other suitable hydrogen bond acceptors other than those recited herein may also be used together with a suitable hydrogen bond donor to form the eutectic solvent.

Similarly, a range of hydrogen bond donors may be used to form the eutectic solvent. In one embodiment, the hydrogen bond donor is selected from the group consisting of an amide, an amino acid, a carboxylic acid, an aromatic acid, an azole, a sugar, a sugar alcohol, and a polyol (including diols and triols), or combinations thereof. In one embodiment, the hydrogen bond donor is selected from the group consisting of a sugar, a polyol, a carboxylic acid, and a carbamide, or combinations thereof.

In one embodiment, the polyol is selected from the group consisting of ethylene glycol, diethylene glycol, triethylene glycol, polyethylene glycol, propylene glycol, polypropylene glycol, and glycerol. In one embodiment, the polyol is glycerol. In one embodiment, the sugar is selected from the group consisting of sucrose, glucose, fructose, lactose, maltose, galactose, xylose, mannose, trehalose, mannitol, sorbitol, inositol, or xylitol, or combinations thereof. In one embodiment, the carboxylic acid is a fatty acid. In one embodiment, the fatty acid is saturated fatty acid. In one embodiment, the carboxylic acid is dodecanoic acid. In one embodiment, the carbamide is selected from the group consisting of urea, 1-methyl-urea, 1,3-dimethylurea, and 1,1-dimethylurea. In one embodiment, the carbamide is urea. In one embodiment, the hydrogen bond donor is selected from the group consisting of glycerol, urea, galactose and dodecanoic acid. It will be appreciated that other suitable hydrogen bond donors other than those recited herein may also be used together with a suitable hydrogen bond acceptor to form the eutectic solvent.

Any one or more of the above hydrogen bond acceptors may be mixed with any one or more of the above hydrogen bond donors to form the eutectic solvent. However, in preferred embodiments, a single hydrogen bond acceptor/donor pair is used. In one embodiment, the eutectic solvent comprises a mixture selected from choline chloride and glycerol, choline chloride and urea, or betaine and glycerol. In one preferred embodiment, the eutectic solvent comprises a mixture of choline chloride and glycerol, or a mixture of choline chloride and urea.

In one preferred embodiment, the eutectic solvent comprises a mixture of choline chloride and glycerol. In another preferred embodiment, the eutectic solvent comprises a mixture of choline chloride and urea. In yet another preferred embodiment, the eutectic solvent comprises a mixture of betaine and glycerol. It will be appreciated that other possible mixtures of hydrogen bond acceptors and donors are contemplated, including those not disclosed herein.

The eutectic solvent comprises any amount of the hydrogen bond acceptor(s) and hydrogen bond donor(s) sufficient to lower the melting point of the solvent mixture to a point that is lower than the melting point of the components individually. There is no limitation placed on the amount of either the hydrogen bond acceptor or hydrogen bond donor present in the eutectic solvent provided a depression in melting point is achieved as understood by the person skilled in the art. Accordingly, the present disclosure encompasses any ratio of hydrogen bond acceptor to hydrogen bond donor that is effective at lowering the melting point of the solvent mixture to a point that is lower than the melting point of either of the components individually.

In one embodiment, the molar ratio of the hydrogen bond acceptor to the hydrogen bond donor is between about 1:5 to about 5:1. The molar ratio of the hydrogen bond acceptor to the hydrogen bond donor may be at least about 1:5, 1:2, 1:1, 2:1, 3:1, 4:1, or 5:1. The molar ratio of the hydrogen bond acceptor to the hydrogen bond donor may be less than about 5:1, 4:1, 3:1, 2:1, 1:1, 1:2 or 1:5. The molar ratio of the hydrogen bond acceptor to the hydrogen bond donor in the eutectic solvent may be in a range provided by any two of these upper and/or lower values, for example between about 1:5 to about 5:1, for example between about 2:1 to about 1:5, or between about 1:1 to about 1:5.

In one embodiment, the eutectic solvent is a deep eutectic solvent (DES), wherein the molar ratio of the hydrogen bond acceptor to the hydrogen bond donor in the eutectic solvent is at or near the eutectic point of the solvent. As used herein, the term “eutectic point” refers to the molar ratio of the components of a given mixture which give the lowest melting point. DES are therefore liquids close to the eutectic composition of the mixture. As understood by the person skilled in the art, deep eutectic solvents are mixtures of hydrogen bond donors and acceptors characterised by a significant depression in melting point compared to that of the individual components. For example, where the eutectic solvent comprises a mixture of choline chloride and glycerol, the molar ratio of choline chloride to glycerol may be about 1:2, which is at or near the eutectic point of the mixture. Similarly, in another example, where the eutectic solvent comprises a mixture of choline chloride and urea, the molar ratio of choline chloride to urea may be about 1:2, which is at or near the eutectic point of the mixture. In yet another example, where the eutectic solvent comprises a mixture of betaine and glycerol, the molar ratio of betaine to glycerol may be about 1:2, which is at or near the eutectic point of the mixture. The eutectic point for a given eutectic solvent can be readily determined by reference to appropriate phase diagrams. An example of such a phase diagram is provided in FIG. 9 for choline chloride: urea (Gilmore et al. Journal of Chemical and Engineering Data, 64(12)5248. It will be appreciated that suitable phase diagrams of possible eutectic solvents described herein can be readily found in literature and/or obtained via melting point analysis as understood by a person skilled in the art.

It will be appreciated that the presence of water and/or additional organic solvents in the eutectic solvent over and above the hydrogen bond donors/acceptors deep eutectic solvents may have undesirable impact on one or more properties of the antimicrobial gel. According to at least some embodiments or examples described herein, the eutectic has a water content (% w/w) of less than about 5, 4, 3, 2, 1, 0.1 or 0.001 based on the total weight of the eutectic solvent. In one embodiment, the eutectic solvent has a water content (% w/w) of between about 0.001 to about 5, between about 0.001 to about 4, or between about 0.001 to about 3, between about 0.001 to about 2, or between about 0.001 to about 1. In one embodiment, the eutectic solvent is substantially free of water (e.g. a DES).

In one embodiment, the deep eutectic solvent is a natural deep eutectic solvent (NADES). NADES are bio-based deep eutectic solvents which are composed of one or more components that are generally non-toxic, such as choline chloride and urea, choline chloride and glycerol and/or betaine and glycerol.

The eutectic solvent may be provided in the antimicrobial gel in an amount effective to form a gel when mixed with a gelling agent described herein. In some embodiments, the concentration of eutectic solvent (% w/w) in the antimicrobial gel is between about 50 to about 99.9 based on the total weight of the antimicrobial gel. In one embodiment, the concentration of eutectic solvent (% w/w) is at least about 50, 55, 60, 65, 70, 75, 80, 85, 90, 95, 98, 99, or 99.9 based on the total weight of the antimicrobial gel. In one embodiment, the concentration of eutectic solvent (% w/w) is less than about 99.9, 99, 98, 95, 90, 85, 80, 75, 70, 65, 60, 55 or 50 based on the total weight of the antimicrobial gel. The % w/w concentration of eutectic solvent in the antimicrobial gel may be a range provided by any two of these upper and/or lower values, for example the concentration of eutectic solvent (% w/w) may be between about 70 to about 99.9, between about 80 to about 99.9 or between about 90 to about 99.9.

According to at least some embodiments or examples, the present inventors have surprisingly found that antimicrobial gels formed using eutectic solvents have high degradation temperatures and/or low vapour pressures meaning they do not dry out over a prolonged period of time. This non-volatility of the antimicrobial gels means that these gels can be used in the open air without risk of evaporation, including at elevated temperatures. When used as wound dressings, this minimizes the need for ongoing reapplication, as the antimicrobial gels retain the eutectic solvent and remain “moist” (i.e. do not dry out) throughout the entire wound treatment period, thus improving overall care for patients.

Furthermore, such little to no evaporation of the eutectic solvent from the gel means the antimicrobial gel can have a longer shelf-life and/or are easily transported. Additionally, the eutectic solvent of the antimicrobial gels can be tailored to allow solubilisation of antimicrobial agents that are not soluble in water. In contrast, conventional water-based gels such as hydrogels evaporate and dry out quickly which necessitates regular removal and reapplication when used as wound dressings which can cause more pain/injury, for example to burns victims. They are also essentially limited to incorporating and delivering water soluble drugs. The antimicrobial gels described herein address these problems.

### Gelling Agent

A gelling agent is mixed with the eutectic solvent to form the gel. As used herein, the term “gelling agent” refers to any substance which forms a gel when in contact with a eutectic solvent as described herein. According to some embodiments or examples, on contact with the eutectic solvent, the gelling agent molecules interact with each other via one or more physical interactions, such as hydrogen bonding, to form a soft physical gel (as opposed to a chemically cross-linked gel). By forming a soft physical gel, antimicrobial agents can be readily incorporated and dispersed therein and readily released topically onto the skin, such as onto a wound. In contrast, chemically cross-linked water-based hydrogels may leave unreacted chemical crosslinking agents on the skin, which may be toxic, requiring costly purification or sterilization procedures. In addition, such water-based gels evaporate and dry out quickly which necessitates regular removal and reapplication which can cause more pain/injury, for example to burns victims.

Any suitable gelling agent may be used to form the antimicrobial gel. In one embodiment, the gelling agent does not require water and/or annealing to form the gel. In one embodiment, the gelling agent is derived from one or more of of algae, plants, animals, fungi, or bacteria. Examples of gelling agents derived from algae include agar, alginate, carrageenan and agarose. Examples of gelling agents derived from plants include pectins. Examples of gelling agents derived from animals include gelatin and keratin. Examples of gelling agents derived from fungi include galactomannan. Examples of gelling agents derived from bacteria include gellan gum and bacterial polysaccharides.

In one embodiment, the gelling agent comprises a polysaccharide (i.e. a carbohydrate polymer). A polysaccharide is a polymeric carbohydrate comprising two or more monosaccharide units bound together by glycosidic linkages. Polysaccharides are widespread in nature and can be found in animals, plants, and bacteria, and can also be synthetically manufactured. In some embodiments, the polysaccharide may be a naturally occurring polysaccharide (e.g. cellulose, bacterial cellulose, agar, or agarose).

In one embodiment, the polysaccharide is selected from the group consisting of cellulose, starch, carrageenan, agarose, agar, alginate, and gellan gum, or a derivative thereof.

In one embodiment, the plant polysaccharide (e.g. derived from plants) is selected from the group consisting of cellulose, starch, guar gum, pectin, locust bean gum, gum arabic, tragacanth and karaya, or a mixture thereof. In one embodiment, the algal polysaccharide (e.g. derived from algae) is selected from the group consisting of carrageenan, agarose, agar and alginate, or a mixture thereof. In one embodiment, the polysaccharide is carrageenan. In one embodiment, the animal derived polysaccharide is chitosan (e.g. from crustacean shells), chitin or gelatin, or a mixture thereof. In one embodiment, the fungal polysaccharide is galactomannan. In one embodiment, the bacterial polysaccharide may be selected from the group consisting of bacterial cellulose, xanthan gum, gellan gum, bacterial alginate, glucans, hyaluronan, succinoglycan, levan, GalactoPol and FucoPol. In one embodiment, the gelling agent is not xanthan gum.

In one embodiment, the gelling agent is a cellulose or derivative thereof. In one embodiment, the gelling agent is an oxidised or partially oxidised cellulose or derivative thereof. Examples of cellulose or derivatives thereof include for example cellulose, partially oxidised cellulose nanofibrils (OCNF), methylcellulose, ethylcellulose, benzyl cellulose, sodium carboxymethylcellulose hydroxyethyl cellulose, hydroxypropyl cellulose, hydroxyethylmethyl cellulose and hydroxypropylmethyl cellulose. Other suitable cellulose derivatives are also described in Tudoroiu et al. Pharmaceuticals (Basel), 2021 Dec. 14(12) 1215. In one embodiment, the cellulose is OCNF. OCNF can be produced via TEMPO-mediated oxidation.

In one embodiment, the cellulose is a bacterial cellulose. The bacterial cellulose may be derived from any number of bacteria, including for example one or more of Aerobacter, Agrobacterium, Pseudomonas, Rhizobium, Alcaligenes, Saecina, and Zoogloea. According to at least some embodiments or examples described herein, Acetobacter xylinum is capable of synthesizing fibrous cellulose extracellularly and, therefore, is able to produce a bacterial cellulose that has been surprisingly identified to be efficient as a gelling agent with eutectic solvents to form the antimicrobial gel. In one embodiment, the bacterial cellulose is derived from Acetobacter xylinum, which may be prepared using the process described in Spicer et al. Biomacromolecules, 2022, 23, 6, 2404-2414. According to some embodiments or examples described herein, this and likely other bacterial celluloses effectively gelled eutectic solvents, including DES's described herein, without requiring additional water to be added.

The gelling agent is provided in the antimicrobial gel in an amount effective to form a gel when in contact with the eutectic solvent. In one embodiment, the concentration of gelling agent (% w/w) in the antimicrobial gel is between about 0.1 to about 10 based on the total weight of the antimicrobial gel. In one embodiment, the concentration of gelling agent (% w/w) is at least about 0.1, 0.2, 0.5, 1, 1.5, 2, 2.5, 3, 3.5, 4, 5, 6, 7, 8, 9 or 10 based on the total weight of the antimicrobial gel. In one embodiment, the concentration of gelling agent (% w/w) is less than about 10, 9, 8, 7, 6, 5, 4, 3.5, 3, 2.5, 2, 1.5, 1, 0.5, 0.2 or 0.1 based on the total weight of the antimicrobial gel. The % w/w concentration of gelling agent in the antimicrobial gel may be a range provided by any two of these upper and/or lower values, for example the concentration of gelling agent (% w/w) may be between about 0.1 to about 5, between about 0.1 to about 4, or between about 0.1 to about 3.

### Additives and Properties

The antimicrobial gel may optionally comprise one or more additives. The one or more additives may be selected from the group consisting of salts, solvents, co-solvents, film forming agents, carriers, colouring agents, penetration enhancers, permeation enhancers, surfactants, humectants, plasticizers, soothing agents or other inactive ingredients, and combinations thereof. The additives may be present in any suitable amount, including, for example, between 1% w/w to about 10% w/w based on the total weight of the antimicrobial gel.

In some instances, the antimicrobial gel or eutectic solvent as described herein may also include, for example, impurities in low amounts, such as in an amount (% w/w) by weight based on the total weight of the antimicrobial gel or eutectic solvent of less than about 5, 4, 3, 2, 1, 0.5, 0.1, 0.01, 0.001, or 0.0001, such as water

Preferably, the antimicrobial gel does not comprise any significant amount of water. However, it is possible that an amount of water may be present in the antimicrobial gel. For example, water may be used to dissolve/suspend the gelling agent and/or antimicrobial agent during the formation of the antimicrobial gels. While according to some embodiments or examples described herein, the antimicrobial gels are dried to remove water, there may be some residual water present in the antimicrobial gel.

In one embodiment, the antimicrobial gel is substantially free of water, and may be considered “substantially anhydrous” or “substantially non-aqueous”. The terms “substantially free of water”, “substantially dry”, “substantially anhydrous” and “substantially non-aqueous” are understood to mean no water is deliberately added and/or retained in the antimicrobial gel. A substantially anhydrous/substantially non-aqueous antimicrobial gel may have a low water content (% w/w) of less than about 5, 4, 3, 2, 1, 0.1 or 0.001 based on the total weight of the antimicrobial gel. In one embodiment, the antimicrobial gel has a water content (% w/w) of between about 0.001 to about 5, between about 0.001 to about 4, or between about 0.001 to about 3, between about 0.001 to about 2, or between about 0.001 to about 1. In contrast to other gels which require high levels of water (such as hydrogels), having a low water content allows for the incorporation of antimicrobial agents that are typically water insoluble. Additionally, antimicrobial gels having low to no water content do not require any special storage or transport conditions unlike water-based hydrogels.

Along with a substantial absence of water, it may also be advantageous if the antimicrobial gel does not comprise any additional organic solvents other than the eutectic solvent used to form the eutectogel. Therefore, in one embodiment, the antimicrobial gel has an additional organic solvent content (% w/w) of less than about 10, 9, 8, 7, 6, 5, 4, 3, 2, 1, 0.1 or 0.001 based on the total weight of the antimicrobial gel. In one embodiment, the antimicrobial gel is substantially free of an additional organic solvent. The additional organic solvent may be methanol, ethanol, ethyl acetate, dimethylsulfoxide (DMSO), phenol, methylene chloride, chloroform, diethyl ether, isopropanol, acetonitrile, tetrahydrofuran, acetone, methyl ethyl ketone, pyridine, and other organic solvents. By avoiding such additional organic solvents, the integrity of the antimicrobial agent may be retained and/or enhanced.

The antimicrobial gel will have an increased viscosity compared to the viscosity of the eutectic solvent prior to contact with the gelling agent. For example, the mixtures of choline chloride and urea, choline chloride and glycerol, and betaine and glycerol, are viscous liquids on their own, but addition of the gelling agent to any one of them results in immediate thickening and increase of viscosity.

In one embodiment, the viscosity of the gel prior to incorporation of the antimicrobial agent may be between 100 Pa·S to about 10,000 Pa·S when measured at a shear rate of 0.1 s⁻¹ at 25° C. Unless otherwise stated, the viscosity of the gel is measured using a rotational rheometer, such as a Discovery Hybrid Rheometer, Model HR-3 (TA instruments). In one embodiment, the viscosity of the gel prior to incorporation of the antimicrobial agent may be less than about 10,000 Pa·S when measured at a shear rate of 0.1 s⁻¹ at 25° C. At higher shear rates (e.g. 1 s⁻¹), the viscosity of the gel prior to incorporation of the antimicrobial agent may be between 100 Pa·S to about 1000 Pa·S at 25° C. By tailoring the amount of gelling agent, the viscosity of the antimicrobial gel can be controlled, including forming firm gels that can act as a bandage over a cut or graze, or as a viscous liquid which can be applied as a gel lotion over a wound, such as a burn or rash.

### Process for Preparing Antimicrobial Gels

The present disclosure also provides a process for preparing an antimicrobial gel, comprising mixing a gelling agent and a eutectic solvent comprising a hydrogen bond acceptor and hydrogen bond donor under conditions effective to form a gel, and wherein the process comprises mixing an antimicrobial agent with the eutectic solvent and gelling agent or contacting the gel with the antimicrobial agent under conditions effective to incorporate the antimicrobial agent into the gel.

The embodiments and examples described above for the antimicrobial gel equally apply to the process for preparing the antimicrobial gel.

In one embodiment, the antimicrobial agent is mixed with the eutectic solvent prior to addition of the gelling agent. In another embodiment, the antimicrobial agent is mixed with the gelling agent prior to addition to the eutectic solvent. It will be appreciated that either mixing arrangement incorporates the antimicrobial agent within the antimicrobial gel.

In one embodiment, the process comprises heating a mixture comprising a hydrogen bond donor and hydrogen bond acceptor under conditions effective to form the eutectic solvent (e.g. as a homogenous eutectic mixture). The hydrogen bond acceptor and hydrogen bond donor may be mixed at a temperature effective to form the eutectic solvent. In one embodiment, the hydrogen bond donor and hydrogen bond accepter are mixed at a temperature (in ° C.) of between about 20 to about 100 to form the eutectic solvent. In one embodiment, the hydrogen bond donor and hydrogen bond accepter are mixed at a temperature (in ° C.) of at least about 20, 30, or 40. In one embodiment, the hydrogen bond donor and hydrogen bond accepter are mixed at a temperature (in ° C.) of less than 100, 90, 80, 70 or 60. The mixing temperature may be a range provided by any two of these upper and/or lower values, for example the hydrogen bond donor and hydrogen bond accepter are mixed at a temperature (in° C.) example between about 30 to about 60, e.g. about 50. According to some embodiments or examples, these low mixing temperatures allow for easy scale up of the synthesis and low-cost manufacturing.

In one embodiment, the gelling agent is suspended in an aqueous solution (such as water) to form a gelling agent solution prior to mixing with the eutectic solvent. The concentration of the gelling agent in the aqueous solution (in % w/w) may be between 0.1 to about 10, for example between about 0.1 to about 5, based on the total weight of the aqueous gelling solution. Where the gelling agent is mixed with the eutectic solvent as a solution, in one embodiment the ratio of eutectic solvent to the gelling agent solution may be between about 1:5 to about 5:1, for example between about 1:2 to about 2:1, for example, about 1:1.

In a related embodiment, the antimicrobial agent is also suspended in an aqueous solution, which may be a separate solution or as part of the gelling agent suspension described above. The antimicrobial agent suspension is either contacted with the antimicrobial gel or eutectic solvent depending on whether it is suspended as a separate solution or as part of the gelling agent suspension. For example, once the eutectic gel is formed, the gel is mixed with an aqueous suspension comprising silver nanoparticles, and dried to form the antimicrobial gel. Silver nanoparticles are typically provided in aqueous solution (that is, the nanoparticles are natively hydrophilic). Owing to this, various challenges had to be overcome to successfully incorporate silver nanoparticles within the non-aqueous environment of the eutectic solvent/gel. Counterintuitively, the present inventors added the silver nanoparticles to the eutectic solvent/gel as an aqueous suspension. Where the gelling agent and/or antimicrobial agent are contacted with the eutectic solvent and/or antimicrobial gel as an aqueous solution, the antimicrobial gel is then dried to remove water, for example to reduce the water content of the antimicrobial gel (% w/w) to less than about 5, 4, 3, 2, 1, 0.1 or 0.001 based on the total weight of the antimicrobial gel. The drying may be any conventional drying process such freeze-drying. Surprisingly, the present inventors have identified that water was not critical to the formation of the eutectogels. According to some embodiments or examples, as a result of the low water content, the antimicrobial gels do not dry out over a prolonged period of time thus minimizing the need for ongoing reapplication when used as a wound dressing, thus improving overall care for patients. In contrast, gels that require water to form, such as hydrogels or xanthan gum-based gels, evaporate and dry out quickly which necessitates regular removal and reapplication which can cause more pain/injury, for example, to burns victims.

In one embodiment, the process comprises incorporating the antimicrobial gel into a container, for example a dispensing tube, jar or punnet. This allows the gel to be applied as a topical cream or gel to the skin.

In another embodiment, while the antimicrobial gel can be applied directly to skin as a topical treatment such as a cream or gel layer, the process may further comprise incorporating the antimicrobial gel on or within a topical dressing. The topical dressing may be any suitable substance capable of forming a protective barrier over a wound on skin. In one embodiment, the topical dressing is selected from the group consisting of a bandage, a wipe, a sponge, a mesh, a gauze, a patch, a pad, tape, or a wrap. In one preferred embodiment, the topical dressing is a wound dressing.

### Compositions and Kits

The antimicrobial gel may be incorporated on or within a topical dressing. Accordingly, the present disclosure also provides a topical dressing comprising the antimicrobial gel.

In one embodiment, the antimicrobial gel is incorporated within the topical dressing or is provided as a coating on a surface of the topical dressing. In one embodiment, the topical dressing is selected from the group consisting of a bandage, a wipe, a sponge, a mesh, a gauze, a patch, a pad, tape, or a wrap. In one embodiment, the topical dressing is a wound dressing.

The antimicrobial gel may also be incorporated into a container, for example a dispensing tube, jar or punnet.

The antimicrobial gel described herein may also be provided as a kit, optionally including instructions for use of the antimicrobial gel (e.g. for treating wounds). That is, the kit can include a description of use of an antimicrobial gel as described herein.

In some embodiments, the kit comprises the components of the antimicrobial gel (e.g. the eutectic mixture and antimicrobial agent), either packaged separately or together. In an embodiment, the hydrogen bond acceptor and hydrogen bond donor may be provided in the kit separately. In some embodiments, the kit may also include a topical dressing. In a preferred embodiment, the kit comprises the antimicrobial gel and topical dressing. The antimicrobial gel may be in a container, such as a dispensing tube, jar or punnet.

The embodiments and examples described above for the antimicrobial gel equally apply to the compositions, topical dressings and kits described herein.

### Methods and Uses of the Antimicrobial Gel

The antimicrobial gel described herein can be topically applied on the skin to treat a variety of skin conditions, including wounds such as burns, grazes and cuts. Accordingly, the present disclosure provides an antimicrobial gel or topical dressing comprising the antimicrobial gel for use in treating and/or preventing a skin condition.

The present disclosure also provides a method of treating and/or preventing a skin condition in a subject in need thereof, the method comprising applying a therapeutically effective amount of the antimicrobial gel or topical dressing comprising the antimicrobial gel to the skin of the subject. The present disclosure also provides use of the antimicrobial gel or topical dressing of for treating and/or preventing a skin condition. The present disclosure also provides use of the antimicrobial gel or topical dressing of for the manufacture of a medicament for treating and/or preventing a skin condition in a subject.

The embodiments and examples described above for the antimicrobial gel and topical dressing equally apply to the methods and uses described herein.

The unique properties of the antimicrobial gel and/or the topical dressing allow the antimicrobial agent to take full effect on the skin while not drying out, thus minimizing the need for ongoing removal and reapplication which can cause more pain/injury, for example, to burns victims. Additionally, and according to at least some embodiments or examples described herein, it has been surprisingly found that the antimicrobial gel and/or topical dressing allows the delivery of water-soluble and water-insoluble antimicrobial agents to the skin of a subject and thereby allow treatment of the skin condition. Therefore, the methods and uses described herein are a safe, painless and/or convenient way of treating and/or preventing a skin condition in a subject.

Unless stated otherwise, it will be understood that the skin condition refers to a skin condition which is responsive to an antimicrobial agent. The antimicrobial gel may be used to prevent and/or treat a skin condition caused by microorganisms, such as bacteria, fungi, viruses and parasites.

In one embodiment, the skin condition is an infection, such as a fungal infection, a bacterial infection, a viral infection, or a parasitic infection.

The skin condition may be an infection of a wound. Additionally, the antimicrobial gel may be used to prevent an infection of a wound that may be susceptible to a bacterial infection, a viral infection, fungal infection, or a parasitic infection.

Non limiting examples of the wound include cuts and lacerations, surgical incisions or wounds, punctures, grazes, scratches, compression wounds, abrasions, friction wounds (e.g. nappy rash, friction blisters), thermal effect wounds (e.g. burns from cold and heat sources, either directly or through conduction, convection, or radiation, and electrical sources), chemical wounds (e.g. acid or alkali bums) or pathogenic infections (e.g. viral, bacterial, parasitic or fungal) including open or intact boils, skin eruptions, blemishes and acne, ulcers, chronic wounds, (including diabetic-associated wounds such as lower leg and foot ulcers, venous leg ulcers and pressure sores), skin graft/transplant donor and recipient sites, immune response conditions, e.g. psoriasis and eczema, stomach or intestinal ulcers, oral wounds, including a ulcers of the mouth, damaged cartilage or bone, amputation wounds and corneal lesions. The wound may be an open wound which can include, but is not limited to, an abrasion, incision, laceration, puncture, avulsion, cut, or other similar injuries.

The antimicrobial gel can be applied to the wound and/or infection following a suitable dosage and treatment regimen. The dosage and administration regimen depend on the nature and condition of the wound and/or infection being treated, the age and condition of the patient, and any prior or concurrent therapy. The antimicrobial gel can be applied at a necessary frequency, for example, once every month, once every other week, once every week, once every other day, once daily, twice daily, three times daily, or four times daily for a suitable period of time. The treatment can be terminated when the wound is recovered. When necessary, the treatment can resume, for example, if a wound recurs. According to some embodiments or examples described herein, the low volatility of the eutectic solvent within the antimicrobial gels means that these gels can be used for a prolonged period of time before the need to reapply the gel to a wound. This minimizes the need for ongoing reapplication, as the antimicrobial gels retain the eutectic solvent and remain “moist” (i.e. do not dry out) throughout the entire wound treatment period, thus improving overall care for patients.

The subject can be a human or a non-human mammal. As used herein, the term “subject” and “patient” are used interchangeably herein and can refer to both human and non-human animals. The term “non-human animals” includes vertebrates, e.g., mammals and non-mammals, such as non-human primates, sheep, dog, cat, horse, cow, rodents (e.g., mice, rats, etc.) and the like. For example, the subject is a human patient. In one embodiment, the subject is a human subject.

The present application claims priority from AU2022901764 filed on 24 Jun. 2022, the entire contents of which are incorporated herein by reference.

The present disclosure may also be defined with reference to one or more of the following numbered paragraphs:
1. An antimicrobial gel, comprising: a) a eutectic solvent comprising a mixture of a hydrogen bond acceptor and hydrogen bond donor; b) a gelling agent; and c) an antimicrobial agent.
2. The antimicrobial gel of paragraph 1, wherein the concentration of antimicrobial agent (mg/g) is between about 0.001 to about 100 based on the total weight of the antimicrobial gel.
3. The antimicrobial gel of paragraph 1 or paragraph 2, wherein the antimicrobial agent is a topical antimicrobial agent.
4. The antimicrobial gel of any one of paragraphs 1 to 3, wherein the antimicrobial agent is selected from the group consisting of antimicrobial nanoparticles, an antibiotic, an antifungal, an antibacterial, an antiparasitic, and an antiviral, or a combination thereof.
5. The antimicrobial gel of paragraph 4, wherein the antimicrobial nanoparticles are metallic nanoparticles, preferably selected from the group consisting of silver, gold, nickel, platinum, palladium, cadmium, zinc, and copper nanoparticles, and combinations thereof.
6. The antimicrobial gel of paragraph 5, wherein the metallic nanoparticles are silver nanoparticles.
7. The antimicrobial gel of any one of paragraphs 4 to 6, wherein the antimicrobial nanoparticles have a mean average particle size (in nm) of between about 1 to about 1000.
8. The antimicrobial gel of any one of paragraphs 1 to 7, wherein the hydrogen bond acceptor is selected from the group consisting of a quaternary ammonium compound, a quaternary phosphonium compound, a quaternary sulfonium compound, a metal salt, an alcohol, and an amino acid.
9. The antimicrobial gel of any one of paragraphs 1 to 8, wherein the hydrogen bond donor is selected from the group consisting of a sugar, a polyol, a carboxylic acid, and a carbamide.
10. The antimicrobial gel of any one of paragraphs 1 to 9, wherein eutectic solvent comprises a mixture selected from choline chloride and glycerol, choline chloride and urea, or betaine and glycerol.
11. The antimicrobial gel of any one of paragraphs 1 to 10, wherein the molar ratio of the hydrogen bond acceptor to the hydrogen bond donor is between about 1:5 to about 5:1.
12. The antimicrobial gel of any one of paragraphs 1 to 11, wherein the eutectic solvent is a deep eutectic solvent (DES), wherein the molar ratio of the hydrogen bond acceptor to the hydrogen bond donor in the eutectic solvent is at or near the eutectic point of the solvent.
13. The antimicrobial gel of any one of paragraphs 1 to 12, wherein the concentration of eutectic solvent (% w/w) is between about 50 to about 99.9 based on the total weight of the antimicrobial gel.
14. The antimicrobial gel of any one of paragraphs 1 to 13, wherein the gelling agent is derived from one or more of of algae, plants, animals, fungi, or bacteria.
15. The antimicrobial gel of any one of paragraphs 1 to 14, wherein the gelling agent comprises a polysaccharide.
16. The antimicrobial gel of paragraph 15, wherein the polysaccharide is selected from the group consisting of cellulose, starch, carrageenan, agarose, agar, alginate, and gellan gum, or a derivative thereof.
17. The antimicrobial gel of any one of paragraphs 1 to 16, wherein the gelling agent is a cellulose or derivative thereof.
18. The antimicrobial gel of paragraph 17, wherein the cellulose is a bacterial cellulose.
19. The antimicrobial gel of any one of paragraphs 1 to 18, wherein the concentration of gelling agent (% w/w) is between about 0.1 to about 10 based on the total weight of the antimicrobial gel.
20. The antimicrobial gel of any one of paragraphs 1 to 19, comprising: a) a eutectic solvent in an amount of between about 80% w/w to about 99.9% w/w; b) a gelling agent in an amount of between about 0.1% w/w to about 5% w/w; and c) an antimicrobial agent in an amount of between about 0.0001% w/w to about 1% w/w, based on the total weight of the antimicrobial gel.
21. The antimicrobial gel of any one of paragraphs 1 to 20, wherein the antimicrobial gel has a water content (% w/w) of less than about 5, 4, 3, 2, 1, 0.1 or 0.001 based on the total weight of the antimicrobial gel.
22. The antimicrobial gel of any one of paragraphs 1 to 21, wherein the antimicrobial gel is for use in treating and/or preventing a skin condition.
23. A process for preparing an antimicrobial gel of any one of paragraphs 1 to 22, comprising: mixing a gelling agent and a eutectic solvent under conditions effective to form a gel, and wherein the process comprises mixing an antimicrobial agent with the eutectic solvent and gelling agent or contacting the gel with the antimicrobial agent under conditions effective to incorporate the antimicrobial agent into the gel.
24. The process of paragraph 23, wherein the antimicrobial agent is mixed with the eutectic solvent prior to addition of the gelling agent or the antimicrobial agent is mixed with the gelling agent prior to addition to the eutectic solvent.
25. The process of paragraph 23 or paragraph 24, wherein the process comprises heating a mixture comprising a hydrogen bond donor and hydrogen bond acceptor under conditions effective to form the eutectic solvent.
26. The process of any one of paragraphs 23 to 25, wherein the hydrogen bond donor and hydrogen bond accepter are mixed at a temperature (in ° C.) of between about 20 to about 100 to form the eutectic solvent.
27. The process of any one of paragraphs 23 to 26, wherein the antimicrobial gel is dried to remove water.
28. The process of any one of paragraphs 23 to 27, further comprising incorporating the antimicrobial gel on or within a topical dressing.
29. The process of paragraph 28, wherein the topical dressing is selected from the group consisting of a bandage, a wipe, a sponge, a mesh, a gauze, a patch, a pad, tape, or a wrap.
30. The process of paragraph 28 or paragraph 29, wherein the topical dressing is a wound dressing.
31. A topical dressing comprising an antimicrobial gel of any one of paragraphs 1 to 22.
32. The topical dressing of paragraph 31, wherein the antimicrobial gel is incorporated within the topical dressing or is provided as a coating on a surface of the topical dressing.
33. The topical dressing of paragraph 31 or paragraph 32, wherein the topical dressing is selected from the group consisting of a bandage, a wipe, a sponge, a mesh, a gauze, a patch, a pad, tape, or a wrap.
34. The topical dressing of any one of paragraphs 31 to 33, wherein the topical dressing is a wound dressing.
35. A method of treating and/or preventing a skin condition in a subject in need thereof, the method comprising applying a therapeutically effective amount of an antimicrobial gel of any one of paragraphs 1 to 22 or a topical dressing of any one of paragraphs 31 to 34 to the skin of the subject.
36. The method of paragraph 35, wherein the skin condition is a wound, a fungal infection, a bacterial infection, a viral infection, or a parasitic infection.
37. Use of an antimicrobial gel of any one of any one of paragraphs 1 to 22 or a topical dressing of any one of paragraphs 31 to 34 for treating and/or preventing a skin condition.
38. Use of an antimicrobial gel of any one of paragraphs 1 to 22 or a topical dressing of any one of paragraphs 31 to 34 for the manufacture of a medicament for treating and/or preventing a skin condition in a subject.

### EXAMPLES

The present disclosure is further described by the following examples. It is to be understood that the following description is for the purpose of describing particular embodiments only and is not intended to be limiting with respect to the above description.

### Example 1: Materials

### Materials

Choline chloride (Sigma, ≥98%); glycerol (Sigma≥99%), Silver nanoparticles (10 nm, 20 nm and 100 nm, 0.02 mg/mL in aqueous buffer (Sigma); Black phosphorous nanoparticles (Sigma); Bacterial cellulose (prepared using protocols outlined in Spicer et al. Biomacromolecules, 2022, 23, 6, 2404-2414).

### Microorganisms

Pseudomonas aeruginosa—ATCC 27853, Methicillin-resistant Staphylococcus aureus (MRSA)—ATCC 70699, Candida albicans—obtained from South Australia Pathology Laboratory.

### Example 2: Preparation of Eutectic Solvents

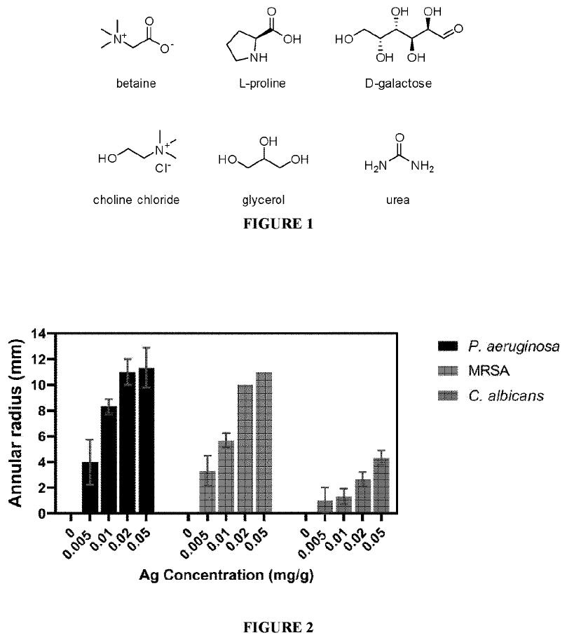

A deep eutectic solvent was made by combining choline chloride (ChCl) and glycerol (Gly) at a molar ratio of 1:2. The molecular structures of each component are shown in FIG. 1. The eutectic mixture was then heated to approximately 50° C. with stirring until a homogenous liquid formed, and was then placed in a freeze drier to remove any residual water.

### Example 3: Preparation of Antimicrobial Gels Comprising Silver Nanoparticles (Varying Concentration)

A 1% w/w suspension of bacterial cellulose in water was combined with an equal weight of the ChCl:Gly eutectic solvent and then dried to achieve a eutectogel with a total concentration of 1 wt % cellulose. Silver nanoparticles (10 nm) in aqueous solution were then mixed with the gel at varying concentrations, and subsequently dried to remove water to obtain antimicrobial gels having the following silver nanoparticle concentrations after drying: 1) 0 mg/g, 2) 0.005 mg/g, 3) 0.01 mg/g, 4) 0.02 mg/g, and 5) 0.05 mg/g, where mg/g means milligrams of silver nanoparticles per gram of antimicrobial gel (i.e. the weight of the gel+nanoparticles).

### Example 4: Preparation of Antimicrobial Gels Comprising Silver Nanoparticles (Varying Size)

A 1% w/w suspension of bacterial cellulose in water was combined with an equal weight of the ChCl:Gly eutectic solvent and then dried to achieve a eutectogel with a total concentration of 1 wt % cellulose. Silver nanoparticles (10 nm, 20 nm or 100 nm) in aqueous solution were then mixed with the gel and subsequently dried to remove water to obtain antimicrobial gels having a silver nanoparticle concentration of 0.05 mg/g having the following particle sizes: 1) 10 nm, 2) 20 nm and 3) 100 nm.

### Example 5: Antimicrobial Gel Testing

### Zone of Inhibition Testing

1. A microbial solution containing P. aeruginosa of optical density (OD) 0.1 was made.
2. 100 uL of the microbial solution was pipetted onto an agar plate (containing 10 mL of agar) and spread across the agar pipette using a sterile cotton bud to create a microbial lawn.
3. Using the largest hole of a 10uL pipette top, well for each sample to be investigated were made in the agar.
4. Each gel was loaded into a well, including a control gel which lacked the Ag NP.
5. The plate was incubated for 24 hours at 25° C.
6. The annular zone of inhibition for each gel was measured. This is the area surrounding the gel-containing wells where no growth is present. Where the rim of the zone of inhibition is comprised of a lighter bacterial lawn (there is some microbial death, but not completely), this was included in the measurement of the zone of inhibition.

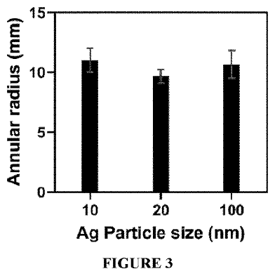

This test was repeated for MRSA and C. albicans, with a total of three replicas for each microorganism. The results of the zone of inhibition testing are shown in FIGS. 2 and 3.

### Serial Dilution Assay

1. 50 mg of each gel containing varying concentrations of Ag NP were placed into 1.5 mL Eppendorf tubes. This was repeated so that for each gel, there was four Eppendorf tubes containing 50 mg of gel each, labelled 3 hours, 6 hours, 24 hours, or 48 hours.
2. A solution containing P. aeruginosa of optical density (OD) 0.1 was made.
3. 100 uL of the microbial solution was pipetted into all the gel-containing Eppendorf tubes and vortexed. 100 uL of the microbial solution in an Eppendorf tube for each time point was used as the control. The samples were left to sit at room temperature.
4. At 3 hours, 900 uL of phosphate buffer solution (PBS) was added to each sample of that time point (total of 6 samples) and vortexed.
5. The serial dilution assay was conducted. First, 180 uL of PBS was pipetted into each well of a single column (8 wells) of a 96-well plate. 20 uL of a sample was pipetted into the first well. Next, 20 uL of the solution in the first well was placed into the second well, and then 20 uL of the solution in the second well was placed into the third well. This was repeated down the column. Then, 5uL from each well was plated in triplicate on agar, and the plate incubated for at least 24 hours at 25° C.
6. Step 5 was repeated for each of the samples at each time point.
7. After incubation, the number of colony-forming units (CFU) for one dilution was counted. Where possible, the dilution with 20-200 colonies was chosen to calculate the number of CFU/mL.
8. CFU/mL was calculated using the following equation:

$$CFU / mL = \frac{Number of colonies \times Dilution factor}{Volume of microbial solution plated}$$

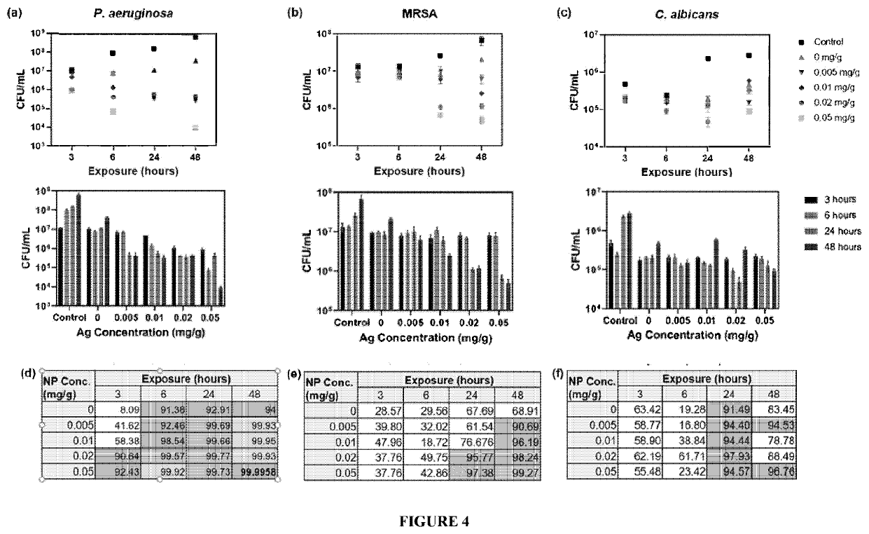

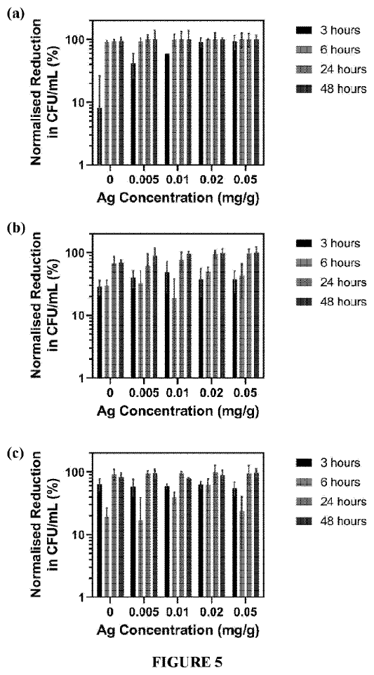

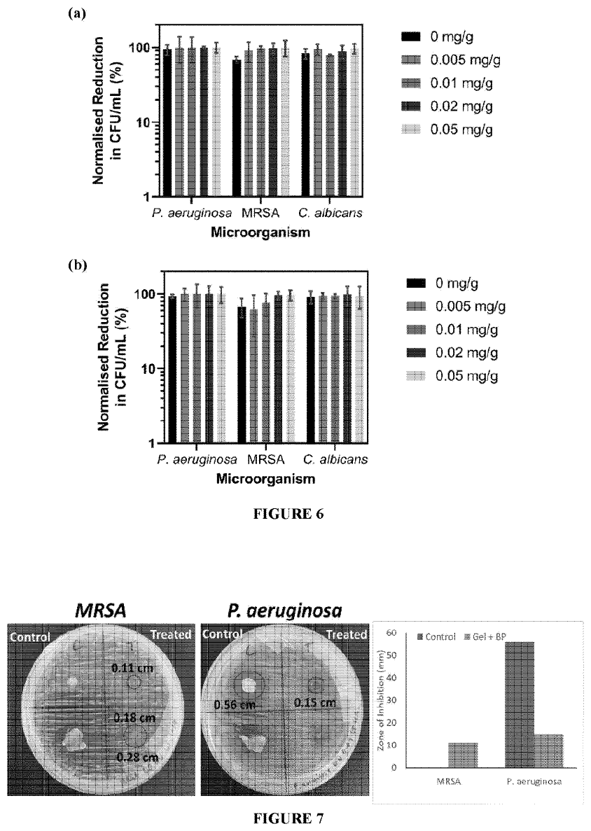

This assay was repeated for MRSA and C. albicans. The results of the serial dilution assays are shown in FIGS. 4, 5 and 6, which demonstrate that the concentration of the Ag NP does impact the antimicrobial efficacy of the Ag-containing gels. In particular, there is a strong positive relationship between Ag NP concentration and the antimicrobial efficacy of the gels. The increase in antimicrobial efficacy as a function of Ag NP concentration appears to plateau at high concentrations (0.02 mg/g, 0.05 mg/g). Overall, the antimicrobial effect of the antimicrobial gels is greater against the bacteria (P. aeruginosa and MRSA) as compared to the fungus (C. albicans). The particle size of the Ag NP does not appear to impact the antimicrobial efficacy of the Ag-containing gels.

### Example 6: Black Phosphorous

Gels were made using the same method described above except that instead of silver nanoparticles, these gels were loaded with black phosphorous nanoparticles. The zone of inhibition (ZOI) of these gels against MRSA and P. aeruginosa are shown in FIG. 7 and Table 1. Two methods of measuring the ZOI were trialled. The black phosphorous-loaded gels showed antimicrobial activity.

TABLE 1
Zone of Inhibition of MRSA and P. aeruginosa around
eutectogels with and without black phosphorus (BP).

| | MRSA (mm) | P. aeruginosa (mm) |
|---|---|---|
| Control A | ND | 56 |
| Control B | ND* | ND* |
| Treatment A | 11 | 15 |
| Treatment B | 18-28** | ND |

ND—not detected
*disruption to bacterial lawn in image due to gel disrupting the lawn when adding to the agar plate (scraped across the agar as it was being removed from the spatula)
**non-uniform zone

The results described herein demonstrate that an antimicrobial gel comprising silver nanoparticles possess antimicrobial activity. Based on this, the antimicrobial gel is expected to be useful for the topical delivery of metallic nanoparticles and other antimicrobial agents, such as water-insoluble drugs, to wounds on the skin.

### Example 7: Antimicrobial Gel Testing

The prepared eutectogel has been tested against three pathogenic microbial species:
1) Pseudomonas aeruginosa—Gram negative bacteria,
2) Methicillin—resistant Staphylococcus aureus (MRSA)—Gram positive bacteria, and
3) Candida albicans—fungal species.

The eutectogel was tested either on its own (“bare”) or when loaded with a range of different antimicrobial substances:
A) silver nanoparticles (AgNPs),
B) black phosphorous nanoflakes (BPNFs),
C) antiseptic octenidine dihydrochloride (Oct),
D) antibiotic tetracycline hydrochloride (Tetra), and
E) antifungal fluconazole (Fluc).

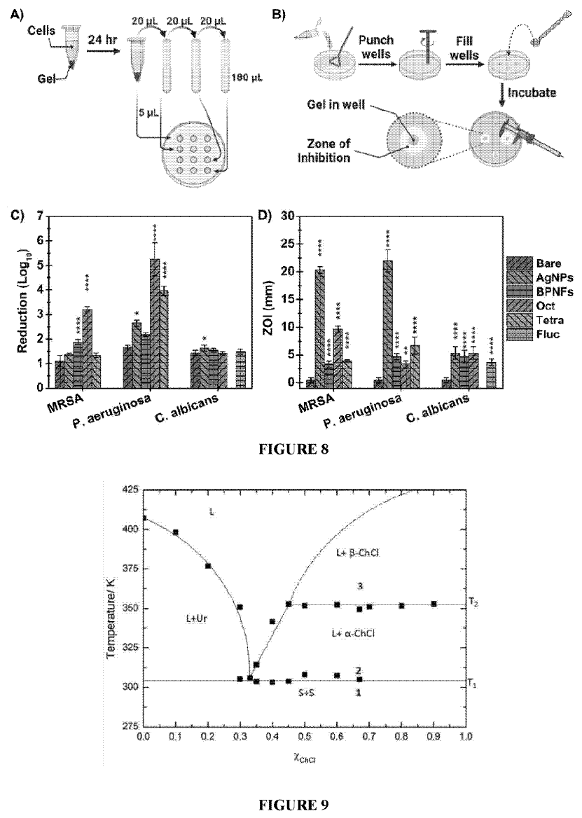

The antimicrobial activity of the bare and loaded eutectogel was tested with two standard methods a) solution-based followed by colony counting, and b) zone of inhibition (ZOI)—both of which are illustrated in FIG. 8.

It was found that the bare gel exhibited bacteriostatic activity based on the solution-based assay but that this activity did not permeate into agar, as shown by the ZOI results. By loading the eutectogels with antimicrobial compounds, an increase in bactericidal activity was seen in both the solution based and ZOI assays. These results, shown in FIG. 8, demonstrate that the eutectogel is an effective carrier/delivery vehicles for a range of antimicrobial substances, including nanoparticles, antiseptics, antibiotics and antifungals. It also demonstrates that these loaded gels were effective against all three of the model antimicrobial species tested which represent a Gram positive, a Gram negative, and a fungal species.

1. An antimicrobial gel, comprising:
a) a eutectic solvent comprising a mixture of at least one hydrogen bond acceptor and at least one hydrogen bond donor;
b) a gelling agent; and
c) an antimicrobial agent.

2. The antimicrobial gel of claim 1, wherein the concentration of antimicrobial agent (mg/g) is between about 0.001 to about 100 based on the total weight of the antimicrobial gel.

3. The antimicrobial gel of claim 1 or claim 2, wherein the antimicrobial agent is a topical antimicrobial agent.

4. The antimicrobial gel of any one of claims 1 to 3, wherein the antimicrobial agent is selected from the group consisting of antimicrobial nanoparticles, an antibiotic, an antifungal, an antibacterial, an antiparasitic, and an antiviral, or a combination thereof.

5. The antimicrobial gel of claim 4, wherein the antimicrobial nanoparticles are metallic nanoparticles, preferably selected from the group consisting of silver, gold, nickel, platinum, palladium, cadmium, zinc, and copper nanoparticles, and combinations thereof, more preferably silver nanoparticles.

6. The antimicrobial gel of any one of claims 1 to 5, wherein the hydrogen bond acceptor is selected from the group consisting of a quaternary ammonium compound, a quaternary phosphonium compound, a quaternary sulfonium compound, a metal salt, an alcohol, and an amino acid, or combinations thereof.

7. The antimicrobial gel of any one of claims 1 to 6, wherein the hydrogen bond donor is selected from the group consisting of a sugar, a polyol, a carboxylic acid, and a carbamide, or combinations thereof.

8. The antimicrobial gel of any one of claims 1 to 7, wherein eutectic solvent comprises a mixture selected from choline chloride and glycerol, choline chloride and urea, or betaine and glycerol.

9. The antimicrobial gel of any one of claims 1 to 8, wherein the eutectic solvent is a deep eutectic solvent (DES), wherein the molar ratio of the hydrogen bond acceptor to the hydrogen bond donor in the eutectic solvent is at or near the eutectic point of the solvent.

10. The antimicrobial gel of any one of claims 1 to 9, wherein the concentration of eutectic solvent (% w/w) is between about 70 to about 99.9 based on the total weight of the antimicrobial gel.

11. The antimicrobial gel of any one of claims 1 to 10, wherein the gelling agent is derived from one or more of of algae, plants, animals, fungi, or bacteria.

12. The antimicrobial gel of any one of claims 1 to 11, wherein the gelling agent comprises a polysaccharide.

13. The antimicrobial gel of claim 12, wherein the polysaccharide is selected from the group consisting of cellulose, starch, carrageenan, agarose, agar, alginate, and gellan gum, or a derivative thereof.

14. The antimicrobial gel of any one of claims 1 to 13, wherein the gelling agent is a cellulose or derivative thereof, preferably bacterial cellulose.

15. The antimicrobial gel of any one of claims 1 to 14, wherein the concentration of gelling agent (% w/w) is between about 0.1 to about 10 based on the total weight of the antimicrobial gel.

16. The antimicrobial gel of any one of claims 1 to 15, comprising:
a) the eutectic solvent in an amount of between about 80% w/w to about 99.9% w/w;
b) the gelling agent in an amount of between about 0.1% w/w to about 5% w/w; and
c) the antimicrobial agent in an amount of between about 0.0001% w/w to about 1% w/w, based on the total weight of the antimicrobial gel.

17. The antimicrobial gel of any one of claims 1 to 16, wherein the antimicrobial gel has a water content (% w/w) of less than about 5 based on the total weight of the antimicrobial gel.

18. The antimicrobial gel of any one of claims 1 to 17, for use in treating and/or preventing a skin condition.

19. A process for preparing an antimicrobial gel of any one of claims 1 to 18, comprising mixing a gelling agent and a eutectic solvent under conditions effective to form a gel, and wherein the process comprises mixing an antimicrobial agent with the eutectic solvent and gelling agent or contacting the gel with the antimicrobial agent under conditions effective to incorporate the antimicrobial agent into the gel.

20. The process of claim 19, further comprising incorporating the antimicrobial gel on or within a topical dressing.

21. A topical dressing comprising an antimicrobial gel of any one of claims 1 to 20.

22. A method of treating and/or preventing a skin condition in a subject in need thereof, the method comprising applying a therapeutically effective amount of an antimicrobial gel of any one of claims 1 to 18, or a topical dressing of claim 21 to the skin of the subject.

23. Use of an antimicrobial gel of any one of claims 1 to 18, or a topical dressing of claim 21 for the manufacture of a medicament for treating and/or preventing a skin condition in a subject.

24. Use of an antimicrobial gel of any one of any one of claims 1 to 18, or a topical dressing of claim 21 for treating and/or preventing a skin condition responsive to an antimicrobial agent.
